(19) United States
(12) Patent Application Publication
(10) Pub. No.: US 2007/0005119 A1
(43) Pub. Date: Jan. 4, 2007

(54) Apparatus and method for the point treatment of a patient by acupuncture and light

(76) Inventor: Steven S. Crohn, Paradise Valley, AZ (US)

Correspondence Address: MICHAEL E. ZALL, TWO YORKSHIRE DRIVE, SUFFERN, NY, 10901, US

(21) Appl. No.: 11/169,371

(22) Filed: Jun. 30, 2005

(51) Int. Cl. A61N 5/06

(52) U.S. Cl. 607/88; 128/907

(57) ABSTRACT

An apparatus for the treatment of a patient at a plurality of treatment points by acupuncture and light treatment. The apparatus includes an enclosure and a source of light within the enclosure. A plurality of flexible conduits for the transmittal of light are provided, each conduit having a first end and a second end. The first end of the conduit is optically connected to the enclosure. A plurality of needles are provided, each needle being suitable for acupuncture and having a first end and a second end having a needle point. The first end of each of the needles is mounted on each of the second ends of the conduits. Each needle is capable of transmitting light therethrough. In use, each needle is implanted into a treatment point of the patient and the light source is activated. Light passes from the light source through the plurality of conduits and needles to the treatment point to simultaneously treat the plurality of treatment points by acupuncture and light.

### BACKGROUND OF THE INVENTION

1. Field of the Invention

This invention relates to the field of acupuncture and the light treatment of a patient, and in particular to an apparatus and method suitable for the simultaneous treatment of a patient at multiple treatment points with acupuncture and light.

2. Description of the Related Art

The present invention relates to a system for the simultaneous treatment of a patient by acupuncture and colorpuncture at multiple treatment points.

In recent years, acupuncture treatment has become very popular. In Eastern Medicine body energy paths are defined as meridian pathways and Jin Shin Jyutsu energy pathways. In acupuncture a localized tissue problem area is treated by needling trigger points (“treatment points”) consisting of nerve endings known to be interconnected along an energy flow path to the localized problem area. One traditional view in acupuncture, acupressure, and reflexology describes the body as a network of meridians or energy channels that pass through the body and its surface to form meridian systems. Therapeutic or treatment points along the meridians may be stimulated, and in some applications unblocked, by various modalities to achieve healthful results potentially for a specific body part of concern, or portion thereof. A view is that such therapies stimulate treatment points that provide a response from the nervous system that provides a therapeutic reaction, such as the release of chemicals in the body for the treatment of a specific body part of concern, or portion thereof.

Conventional acupuncture uses needles implanted at selected acupuncture points in the patient's body in order to treat and/or cure certain types of disorders. The stimulation of these acupuncture points has also been accomplished by applying electric current, pressure, ultra-sound, heat or light. There are numerous known ways of detecting such active treatment points, e.g., by detecting an area of reduced skin impedance.

The needles used in acupuncture are generally metallic needles having on one end a needle point that is implanted into the affected part of a patient's body and on the other end a needle holder. The method of inserting the needle varies depending on the symptoms and the locations of diseased parts. For example, one technique involves implanting a needle into a diseased treatment point or an effective treatment point separate from the diseased part. Another technique involves implanting the needle into an effective treatment point and also burning moxa at such treatment point to apply heat to the treatment point.

One type of acupuncture needle has a resistance heating body at the needle point, which is heated by passing an electric current through it. The needle is complex in structure and expensive and has a drawback of causing side effects due to heat radiating from the entire needle.

Other methods of therapeutic treatment include electromagnetic stimulation of sensory nerves or acupuncture points and focuses on stimulation of nerve tracts for the purpose of promoting release of natural opiates or pain pathway blocks through gating mechanisms. Various forms of such stimulations have been tried such as the application of voltages or currents to acupuncture needles, transcutaneous electrical nerve stimulation (TENS), pulsed magnetic field stimulation, local application of heat or cold, use of light radiation, and magnetic therapy.

Administering transcutaneous energy in the form of light and/or electrical current is generally known to be particularly useful in alleviating certain types of pain. Light or optical therapy to treatment points is well known. Traditionally such a modality of therapeutic treatment causes the stimulation of the treatment points. Some light systems have incorporated coherent light, such as lasers, as a therapeutic modality. Other therapeutic light modalities have been used, such as non-coherent light, provided by devices incorporating light-emitting diodes (LEDs) or other such elements for visible light application, including infrared light.

While there are known devices for the stimulation of treatment points by means of sources of coherent light—lasers, it is also known to stimulate such acupuncture points by means of an incandescent lamp which is focused on the surface of the skin corresponding to an acupuncture point or points. However, with conventional light radiating or optical therapy, while it is easy to apply light onto the surface of the diseased part, the target for acupuncture treatment within the body cannot be radiated with light without cutting into and exposing the affected part.

Colorpuncture, a form of optical or light therapy, is a holistic healing procedure claimed to be invented by a German scientist and naturopath named Peter Mandel. Allegedly he has conducted over 25 years of intensive empirical research to develop this unique system of healing. Colorpuncture involves focusing colored light on acupuncture (and other) treatment points on the skin in order to energize powerful healing impulses in our physical and energy bodies, see, http://www.colorpuncture.com/whatis.html.

In a colorpuncture treatment, frequencies of colored light are focused on the skin using a hand-held acu-light tool with specially designed, hand-made interchangeable glass rods which emit different colors of light through a focused tip. Each color consists of different wavelength frequencies of light and therefore communicates different energetic information. Treatments include a specific set of points in a sequence using a prescribed pattern of colors. As the light is absorbed by the skin and transmitted along energetic pathways or meridians deep into the body, it stimulates intra-cellular communication which supports healing.

Some examples of existing prior art include the following:

U.S. Pat. No. 4,232,678 to Skovajsa describes a device for the local treatment of a patient by acupuncture or auriculotherapy. Instead of needles, a treatment head is passed close to the body of the patient. The head includes an infra-red laser diode being excitable recurrently and in a controlled manner. The recurrence frequency is selectable among a plurality of discrete frequencies, each of which may be finely adjusted.

U.S. Pat. No. 4,535,784 to Rohlicek, et al describes an apparatus for stimulating and locating acupuncture points by light radiation in the visible light range or in the infrared range for medical purposes.

U.S. Pat. No. 5,024,236 to Shapiro describes a compact, portable photoprobe assembly for locating acupuncture points and a light emitting diode to stimulate acupuncture points as well as injury sites. The probe and light emitting diode are controlled by an internal circuit which operates to provide a visual indication of the precise location of the desired treatment area via an impedance variation procedure as well as to pulse the light emitting diode at the desired frequency. A power supply is also contained within the assembly to render it completely portable.

U.S. Pat. No. 5,250,068 to Ideguchi, et al. describes an acupuncture needle that comprises a needle body with an optical fiber for transmitting light from a light source over nearly the entire length of the needle, and a needle point attached to the front end of the optical fiber. A light collecting lens is provided for applying light from the light source onto the end surface of the optical fiber so that the light can be transmitted through the optical fiber. The needle point inserted into the affected part of the body collects light from the light source and radiates light or heat against the affected part to produce improved remedial effects.

U.S. Pat. No. 5,644,436 to Smith discloses a method and device for performing laser therapy. The method includes providing a laser source for emitting a laser light, diagnosing an afflicted area of the patient, delivering the laser light to the afflicted area for at least one treatment cycle, monitoring the afflicted area after the treatment cycle has been completed, and repeating the steps of diagnosing and delivering the laser light to the afflicted area based on the monitoring step.

U.S. Pat. No. 5,843,074 to Cocilovo discloses a non-coherent pulsed and colored light stimulation device used for a therapeutic effect. Pulsed/colored light is applied to local areas, or acupuncture macro or micro systems, by means of a small diameter optic fiber housed in a pen-like handpiece which makes application comfortable and precise. The light source is an adjustable rate strobe with a housing attached to the front which allows color gel slides to be interchanged. The light passes through the slides and through a length of jacketed flexible optic fiber where it becomes visible again at the radiant tip of the handpiece.

U.S. Pat. No. 6,302,900 to Riggs discloses a holistic method of treating injured or pathologic tissue, e.g., acupuncture points, with a laser.

U.S. Pat. No. 6,306,160 to Nidetzky discloses a soft laser unit and a skin resistance measuring device for localizing acupuncture points. The unit additionally contains a biostimulation or therapy laser unit for laser acupuncture. The laser unit can be used in continuous operation to produce a “laser shower” without touching the skin.

U.S. Pat. No. 6,416,531 to Chen discloses the application of light at plural treatment sites within a tumor to increase the efficacy of light therapy. A plurality of optical fibers are inserted into the interior of a tumor in a spaced-apart array so that the optical fibers are arranged in a pattern.

U.S. Pat. No. 6,520,903 to Yamashiro discloses an electromagnetic stimulation device that generates combined repetitive pulses of full spectrum light and magnetic field to promote propagation of energy and pain relief in the body.

U.S. Pat. No. 6,641,599 to Peterson, et al. discloses systems of therapeutic treatment (light treatment, acupuncture).

US Patent Application Publication No. 2002/0143373 to Courtnage, et al discloses a therapeutic device for the application to the body. The energy is applied by the action of photon-emitting diodes in combination with transcutaneous electrical stimulators. A shapable housing contains the energy sources and the power grid and is selectively moldable with memory retention to retain a given shape.

As shown in much of the prior art, there are no known systems suitable for the simultaneous treatment of a patient at multiple treatment points, e.g., acupuncture treatment points, with acupuncture and light. The invention described and claimed herein is directed to such an apparatus and method.

### OBJECTS AND SUMMARY OF INVENTION

It is an object of this invention to provide an apparatus and method for the simultaneous treatment or stimulation of acupuncture points by acupuncture and light radiation.

It is another object of the present invention to provide a therapeutic method which can reach deep into tissues yet not harm the tissues, as in the conventional systems.

It is yet another object of this invention to provide an apparatus that is inexpensive and permits the simultaneous treatment of a plurality of treatment points by acupuncture and light to provide an enhanced treatment modality.

All of the foregoing objects as well as others are achieved by an apparatus for the treatment of a patient at a plurality of treatment points by acupuncture and light treatment. The apparatus includes an enclosure and a source of light within the enclosure. A plurality of flexible conduits for the transmittal of light are provided, each conduit having a first end and a second end. The first end of the conduit is optically connected to the enclosure. A plurality of needles are provided, each needle being suitable for acupuncture and having a first end and a second end having a needle point. The first end of each of the needles is mounted on each of the second ends of the conduits. Each needle is capable of transmitting light through the needle point. In use, each needle is implanted into a treatment point of the patient and the light source is activated. Light passes from the light source through the plurality of conduits and the needle points to the treatment points to simultaneously treat the plurality of treatment points by acupuncture and light.

The foregoing objects are also achieved by the method of this invention for treating a patient by acupuncture and light. The method includes locating a plurality of treatment points on a patient, preferably acupuncture treatment points. A plurality of acupuncture needles are provided each capable of transmitting light therethrough, Additionally the method further includes providing a light source for transmitting light through each of the needles, activating the light source to transmit light through each of the needles through the needle point and implanting the plurality of acupuncture needles into the plurality of treatment points located. The plurality of treatment points are each simultaneously treated by light and acupuncture.

Further objects, features and advantages of the invention will become apparent from a consideration of the following description and the appended claims when taken in connection with the accompanying drawings.

### BRIEF DESCRIPTION OF THE DRAWINGS

For a more complete understanding of the present invention, reference may be had to the following description of the exemplary preferred embodiments of the present invention considered in connection with the accompanying drawings, of which:

FIG. 1 is a schematic view of one embodiment of the apparatus of this invention for the treatment of a patient at a plurality of treatment points by acupuncture and light treatment.

FIG. 2 is a side view of the acupuncture needle used in the apparatus depicted in FIG. 1;

FIG. 3 is highly magnified axial cross-sectional view of the acupuncture needle used in the apparatus depicted in FIG. 1;

FIG. 4 is a very highly magnified axial cross-sectional view taken around point A of the acupuncture needle depicted in FIG. 3; and

FIG. 5 is a schematic view of a patient depicting the use of this invention at various acupuncture treatment points on the body.

### DETAILED DESCRIPTION OF THE INVENTION

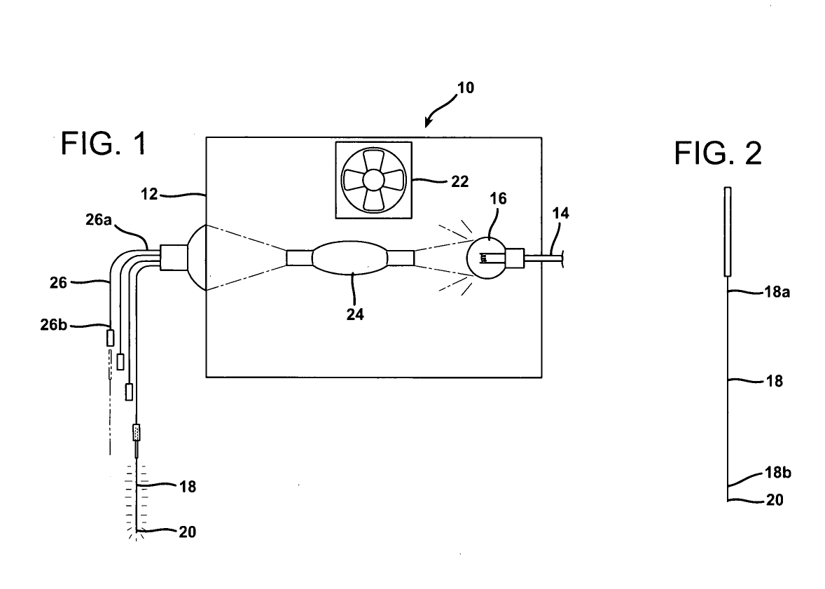

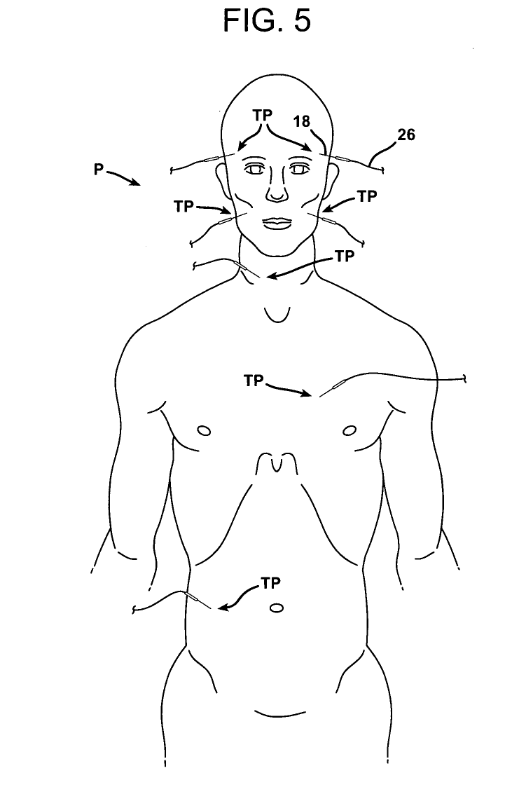

Referring to FIG. 1-5, the apparatus 10 is used for the treatment of a patient P at a plurality of treatment points TP by acupuncture and light treatment. Preferably, energy flow, acupuncture meridians and treatment points TP are generally those defined in Eastern medicine. Another preferred aspect of this invention comprises treating neurological malfunctioning sensory and motor nerve treatment points TP. FIG. 5 shows treatment points TP that are acupuncture points and wherein the paths of energy flow paths are called meridians in accordance with the Eastern acupuncture school of philosophy and practice.

Referring to FIG. 1, the apparatus includes an enclosure or light box 12. The light box 12 may be of any convenient shape and made of any suitable material. It can be a stationary box suitable for placement on a table or shelf close to the patient or may be hand held for easy mobility. The enclosure 12 is provided with a means to access power through an external power cord 14 or may have an internal portable power source such as a battery (not shown).

A source of light 16 is provided within the enclosure 12. It is often desirable, and in fact preferred to treat the treatment points TP with different colors of light treatment. Thus, the source of light 16 may be capable of providing numerous different colors of light with or without the assistance of fiber optic needles 18 of differing colors.

In a colorpuncture treatment, frequencies of colored light are focused on the skin using a hand-held acu-light tool with specially designed, hand-made interchangeable glass rods which emit different colors of light through a focused tip. Each color consists of different wavelength frequencies of light and therefore communicates different energetic information. Treatments include a specific set of points in a sequence using a prescribed pattern of colors. As the light is absorbed by the skin and transmitted along energetic pathways or meridians deep into the body, it stimulates intra-cellular communication which supports healing.

It has been postulated that different colors have differing effects on the body:

Red-stimulates the sensory nervous system, sight, hearing, touch, taste, smell; builds platelets; counter agent for burns from x-rays;

Orange-lung and thyroid builder; stomach stimulant; bone builder;

Yellow-stimulates the intestines; increases bowel movements;

Lemon-bone builder; brain stimulant.

There are numerous other colors of light, e.g., turquise, blue, indigo, violet, purple, magenta, scarlet, each having several different physiological effects on the body.

The apparatus 10 is capable of generating numerous light colors for transmittal through one or more needles 18 for treatment of the patients P treatment points TP. This may be done with a combination of fiber optic needles 18 having needle points 20 of differing predetermined colors and colored prisms, a color wheel, that filter the light source 16.

It may be preferred for optimum treatment that the transmittal of heat to the treatment point TP be minimized. Therefore, in a preferred embodiment a cooling means, e.g., exhaust fan 22 may be installed within the enclosure 12 to remove heat from the enclosure and light being transmitted to the treatment point TP. Additionally, the light beam from the light source 16 can be transmitted through a vacuum tube 24 covered with a heat conducting metal sleeve to assist in dispersing the heat generated by the light source 16.

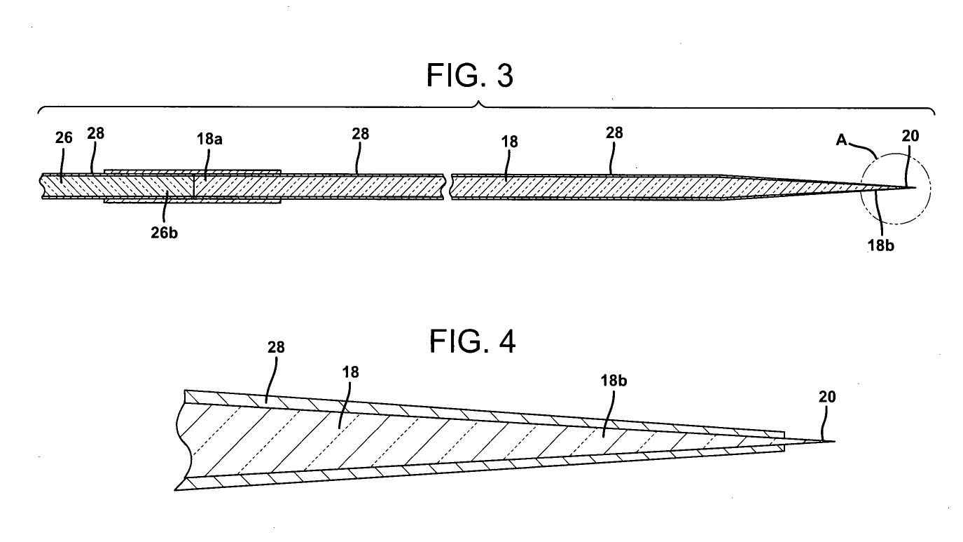

Referring to FIGS. 1-5, a plurality of flexible conduits 26 are provided for the transmittal of light to the treatment point TP. Each conduit 26 has a first end 26a and a second end 26b ( FIG. 3). The first end 26a is optically connected to the enclosure 12 such that light can pass through the conduit 26, such as a fiber optic conduit or fiber 26.

A plurality of needles 18 are provided. Each needle 18 is suitable for acupuncture. The needle 18 has a first end 18a and a second end 18b having a needle point 20. The first end 18a of each of the needles 18 is mounted on each of the second ends 26b of the conduits 26. Each needle 18 is capable of transmitting light through the needle 18 to the needle point 20. The light then passes through the needle point 20. Preferably the needle 18 is a fiber optic conduit or fiber.

The means for mounting the needle end 18a on the end of the conduit 26b can be by a sleeve that fits onto the end of the needle end 18a. Optionally, the conduit 26 and needle 18 can be a continuous fiber optic fiber that terminates in a needle point 20 that can transmit light. Preferably, the needle 18 and/or conduit 26 are coated with a protective coating 28, e.g., a polymer. A preferred polymer is KEVLAR® from DuPont. This coating 28 both protects the needle 18 and contains and concentrates the light within the needle 18. At the end of the needle 18b the coating is removed to permit the optimum transmission of light from the needle point 20 into the treatment point TP in which the needle is implanted.

After implantation of the plurality of needles into the desired treatment points TP of the patient P, the light source 16 is activated. Optionally, the light source 16 can be activated prior to implantation, the pin-point light emanating from the needle point 20 assisting in locating the exact treatment point TP.

Upon implantation of the needle points 20 and activation of the light source 16, the light passes through the conduits 26 and needles 18 to simultaneously treat the plurality of treatment points by needle acupuncture and light.

By the use of the term “light” herein it is meant to include, coherent light, e.g., laser light, non-coherent light, provided by devices incorporating light-emitting diodes (LEDs) or other such elements for visible light application, predetermined colored light or infrared light.

Preferably, fiber optic needles 18 are used with each needle 18 having a predetermined color so that light of a predetermined wavelength (color) is transmitted into the treatment point TP. During a treatment session the predetermined colors of light transmitted to the treatment point TP can all be the same, for example for the treatment of one type ailment or complaint, or can each differ for the treatment of many type ailments or complaints simultaneously.

Thus, the apparatus 10 of this invention can be used to simultaneously treating a patient P by needle acupuncture and light. Referring to FIGS. 1-5, the method of treatment, in its preferred embodiment, comprises:

locating a plurality of treatment points TP on a patient P;

providing a plurality of fiber optic acupuncture needles 18, each capable of transmitting light through the needle 18 to the needle point 20, the needle 18 having a first end 18a and a second end 18b;

providing a light source 16;

providing a plurality of flexible fiber optic conduits 26 for optically connecting the light source 16 with each of the first ends 18a of the needles 18;

activating the light source 16 to transmit light through each of the conduits 26 and through each of the needles 18 to and through the needle point 20;

implanting the plurality of acupuncture needles 18 in the plurality of treatment points TP located;

wherein the plurality of treatment points TP are each simultaneously treated by light and acupuncture.

In use, as the light is repetitively full-reflected at the boundary surface of the needle 18 it travels through the fiber optic core and converges on the transparent needle point 20. The needle point 20 embedded in the body of a patient collects light in a localized portion in the body, i.e., the treatment point TP, and, depending on the characteristic of the light source, can increase the temperature of (no cooling) or merely radiate light onto the affected part in the body (cooling). In this way, the diseased part TP is appropriately stimulated by heat and/or light to promote curing by stimulated physiological action.

Various types of optical fibers are available for use in this invention, such as quartz fiber, multicomponent glass fiber and plastic fiber. Quartz fiber is probably preferred because it has the least transmission loss among these optical fibers. The quartz optical fiber contains quartz as a major component with a germanium oxide and other components added to increase the refractive index.

The needle point 20 requires sufficient strength to be inserted smoothly into the treatment point TP. To avoid stress concentration at the tip, the needle point 20 has the tip rounded with a radius of the order of microns. The material of the needle point 20 is preferably a transparent quartz that can transmit light and needs to be resilient and not brittle.

The needle body 18 is made to various sizes with the diameter d ranging from 0.1 mm to 0.5 mm and the whole length 1 ranging from 50 mm to 300 mm so that an appropriate needle size can be chosen according to the symptoms and the locations of diseased parts. The needle 18 is preferably designed for only a single use.

The light source 16 may include, for example, visible light, infrared rays and laser beam, and an appropriate light and colors can be selected according to the symptoms and the location of diseased part.

The acupuncture needle 18 can be manipulated by an acupuncturist in much the same way as a conventional acupuncture needle in sticking it in the treatment points TP. The light from the light source 16, for example, visible light rays, passes through the end of the needle point 20. The rays of light travel through the core of the optical fiber needle 18 while being reflected by the boundary surface between the coating 28 until they reach the transparent needle point 20. The optical fiberneedle 18 transmits light from the light source 16 to the needle point 20 with minimal loss. The light may also be applied as heat energy to heat the desired part TP.

The temperature of the needle point 20 and the color of the light is adjusted to an appropriate values and colors according to the object of the treatment by selecting a desired light source and cooling effect. In this way, the diseased part in the body is treated with heat and light to stimulate physiological action for improved remedial effects.

A preferred length of flexible fiber optic conduit 26 is from six to eight feet, however not limited to that length. Since there is very little light intensity loss, the length is therefore not limited by intensity variables. The diameter of the jacketed optic fiber conduit 26 in a preferred embodiment is 2.20 mm for micro work but may be 3-4 mm. or larger.

In addition to stimulating acupuncture points, the apparatus of this invention can be used to directly stimulate various wounds and injuries to promote healing and alleviate pain. Examples of such injuries are contusions, scratches, and various open wounds. Treatment of these wounds can be accomplished by stimulating the wound with light radiation and acupuncture for a period of time ranging from one or two minutes to as many as ten minutes several times daily, or as needed, to relieve pain and promote healing. It should be noted that prior to receiving such therapy, the subject should receive appropriate medical therapy for the condition. The inventive therapy is used only for promotion of healing and alleviation of pain.

The apparatus of this invention may be advantageously used therapeutic treatment in hospitals, in ambulatory treatments, and even of treatments of a patient in the home.

The foregoing constitutes a description of specific embodiments showing how the invention may be applied and put into use. These embodiments are only exemplary. The invention in its broadest, and more specific aspects, is further described and defined in the claims which now follow.

These claims, and the language used therein, are to be understood in terms of the variants of the invention which have been described. They are not to be restricted to such variants, but are to be read as covering the full scope of the invention as is implicit within the invention and the disclosure.

What is claimed is:

1. An apparatus for treatment of a patient at a plurality of treatment points by acupuncture and light treatment, the apparatus comprising:
an enclosure;
a source of light within the enclosure;
a plurality of flexible conduits for the transmittal of light, each conduit having a first end and a second end, the first end optically connected to the enclosure; and
a plurality of needles, each needle suitable for acupuncture and having a first end and a second end having a needle point, the first end of each of the needles mounted on each of the second ends of the conduits, each needle capable of transmitting light through the needle point;
wherein when each needle is implanted into a treatment point of the patient and the light source is activated, light passes from the light source through the plurality of conduits and needles to simultaneously treat the plurality of treatment points by acupuncture and light.

2. The apparatus of claim 1, further comprising a cooling means within the enclosure for cooling the enclosure.

3. The apparatus of claim 1, wherein each flexible conduit is a fiber optic conduit.

4. The apparatus of claim 1, wherein each needle is a fiber optic needle.

5. The apparatus of claim 1, wherein each needle is a fiber optic needle of a predetermined color.

6. The apparatus of claim 1, wherein each needle is a fiber optic needle of a predetermined color, the apparatus having a plurality of needles with a plurality of predetermined colors.

7. An apparatus for treatment of a patient at plurality of treatment points by acupuncture and light treatment, the apparatus comprising:
an enclosure;
a source of light within the enclosure;
a plurality of flexible fiber optic conduits for the transmittal of light, each conduit having a first end and a second end, the first end optically connected to the enclosure; and
a plurality of fiber optic needles, each needle suitable for acupuncture and having a first end and a second end having a needle point, the first end of each of the needles mounted on each of the second ends of the conduits, each needle capable of transmitting light of a predetermined color through the needle point;
wherein when each needle is implanted into a treatment point of the patient and the light source is activated, light passes from the light source through the plurality of conduits and the plurality of needles to simultaneously treat the plurality of treatment points by acupuncture and light.

8. A method of treating a patient by acupuncture and light comprising:
locating a plurality of treatment points on a patient;
providing a plurality of acupuncture needles, each with a needle point and each being capable capable of transmitting light through the needle point;
providing a light source for transmitting light through each of the needles to the needle point;
activating the light source to transmit light through each of the needle points;
implanting the plurality of acupuncture needle points into the plurality of treatment points located;
wherein the plurality of treatment points are each simultaneously treated by light and acupuncture.

9. A method of treating a patient by acupuncture and light comprising:
locating a plurality of treatment points on a patient;
providing a plurality of fiber optic acupuncture needles, each needle having a first end and a second end having a needle point capable of transmitting light through the needle point;
providing a light source;
providing a plurality of flexible fiber optic conduits for optically connecting the light source with each of the first ends of the needles;
activating the light source to transmit light through each of the conduits and through each of the needle points;
implanting the plurality of acupuncture needles into the plurality of treatment points located;
wherein the plurality of treatment points are each simultaneously treated by light and acupuncture.

10. The method of treating a patient by acupuncture and light of claim 8, wherein the treatment points are acupuncture treatment points.

11. The method of treating a patient by acupuncture and light of claim 9, wherein the treatment points are acupuncture treatment points.
